SEQUENCE LISTING (6 sequences)

SEQ ID NO: 1 - DNA, 1113 bp, Cricetulus griseus
atggtggtggtggcagcggcgccgagcgcggccacggcggccccgaaagtactgcttctatcgggccagcccgccgcggacggccgggcgctgccactcatggttccaggctcgcgggcagcagggtccgaggcgaacggggcgccacaggctcgcaagcggcagcgcctcacgcacctgagcccggaggagaaggcgctgcggaggaaactgaaaaacagagtagcagcgcagactgcccgagatcgaaagaaagcccggatgagcgagctggaacagcaagtggtggatttggaagaagagaaccaaaaacttctgttagaaaatcagcttttgagagagaaaactcatggccttgtaattgagaaccaggagttaagaactcgcttgggaatggatgtgctgactactgaagaggctccagagacggagtccaagggaaatggagtaaggccggtggccgggtctgctgagtccgcagcaggtgcaggcccagttgtcacctccccagaacatcttcccatggattctgacactgttgactcttcagactccgagtctgatatccttttgggcattctggacaagttggaccctgtcatgtttttcaaatgtccatccccagagtctgccaatctggaggaactcccagaggtctacccaggacctagttccttaccagcctccctttctctgtcagtggggacctcatcagccaagctggaagccattaatgaactcattcgctttgaccatgtatacaccaagcctctagtcttagagatcccttctgagacagagagtcaaactaatgtggtagtgaaaattgaggaagcacctctcagctcttcagaggaggatcaccctgaattcattgtctcagtgaagaaagaacctttggaagaagacttcattccagagccgggcatctcaaacctgctttcatccagccactgtctgaaaccatcttcctgcctgctggatgcttatagtgactgtggatatgagggctccccttctcccttcagtgacatgtcttctccacttggtatagaccattcttgggaggacacttttgccaatgaactctttccccagctgattagtgtctaa

SEQ ID NO: 2 - protein, 370 aa, Cricetulus griseus
MVVVAAAPSAATAAPKVLLLSGQPAADGRALPLMVPGSRAAGSEANGAPQARKRQRLTHLSPEEKALRRKLKNRVAAQTARDRKKARMSELEQQVVDLEEENQKLLLENQLLREKTHGLVIENQELRTRLGMDVLTTEEAPETESKGNGVRPVAGSAESAAGAGPVVTSPEHLPMDSDTVDSSDSESDILLGILDKLDPVMFFKCPSPESANLEELPEVYPGPSSLPASLSLSVGTSSAKLEAINELIRFDHVYTKPLVLEIPSETESQTNVVVKIEEAPLSSSEEDHPEFIVSVKKEPLEEDFIPEPGISNLLSSSHCLKPSSCLLDAYSDCGYEGSPSPFSDMSSPLGIDHSWEDTFANELFPQLISV

SEQ ID NO: 3 - DNA, 2013 bp, Homo sapiens
atgggggagccggctggggttgccggcaccatggagtcaccttttagcccgggactctttcacaggctggatgaagattgggattctgctctctttgctgaacttggttatttcacagacactgatgagctgcaattggaagcagcaaatgagacgtatgaaaacaattttgataatcttgattttgatttggatttgttaccttgggagtcagacatttgggacatcaacaaccaaatctgtacagttaaagatattaaggcagaaccccagccactttctccagcctcctcaagttattcagtctcatctcctcggtcagtggactcttattcttcaactcagcatgttcctgaggagttggatttgtcttctagttctcagatgtctcccctttccttatatggtgaaaactctaatagtctctcttcaccggagccactgaaggaagataagcctgtcactggttctaggaacaagactgaaaatggactgactccaaagaaaaaaattcaggtgaattcaaaaccttcaattcagcccaagcctttattgcttccagcagcacccaagactcaaacaaactccagtgttccagcaaaaaccatcattattcagacagtaccaacgcttatgccattggcaaagcagcaaccaattatcagtttacaacctgcacccactaaaggccagacggttttgctgtctcagcctactgtggtacaacttcaagcacctggagttctgccctctgctcagccagtccttgctgttgctgggggagtcacacagctccctaatcacgtggtgaatgtggtaccagccccttcagcgaatagcccagtgaatggaaaactttccgtgactaaacctgtcctacaaagtaccatgagaaatgtcggttcagatattgctgtgctaaggagacagcaacgtatgataaaaaatcgagaatccgcttgtcagtctcgcaagaagaagaaagaatatatgctagggttagaggcgagattaaaggctgccctctcagaaaacgagcaactgaagaaagaaaatggaacactgaagcggcagctggatgaagttgtgtcagagaaccagaggcttaaagtccctagtccaaagcgaagagttgtctgtgtgatgatagtattggcatttataatactgaactatggacctatgagcatgttggaacaggattccaggagaatgaaccctagtgtgggacctgcaaatcaaaggaggcaccttctaggattttctgctaaagaggcacaggacacatcagatggtattatccagaaaaacagctacagatatgatcattctgtttcaaatgacaaagccctgatggtgctaactgaagaaccattgctttacattcccccacctccttgtcagcccctaattaatacaacagagtctctcaggttaaatcatgaacttcgaggatgggttcatagacatgaagtagaaaggaccaagtctagaagaatgacaaataatcaacagaaaacccgtattcttcagggtgttgtggaacagggctcaaattctcagctgatggctgttcaatacacagaaaccactagtagtatcagcaggaactcagggagtgagctacaagtgtattatgcttcacccagaagttatcaagacttttttgaagccatccgcagaaggggagacacattttatgttgtgtcatttcgaagggatcacctgctgttaccagctaccacccataacaagaccacaagaccaaaaatgtcaattgtgttaccagcaataaacataaatgagaatgtgatcaatgggcaggactacgaagtgatgatgcagattgactgtcaggtgatggacaccaggatcctccatatcaaaagttcgtcggttcctccttacctccgagatcagcagaggaatcaaaccaacaccttctttggctcccctcccgcagccacagaggcaacccacgttgtcagcaccatccctgagtcattacaatag

SEQ ID NO: 4 - protein, 670 aa, Homo sapiens
MGEPAGVAGTMESPFSPGLFHRLDEDWDSALFAELGYFTDTDELQLEAANETYENNFDNLDFDLDLLPWESDIWDINNQICTVKDIKAEPQPLSPASSSYSVSSPRSVDSYSSTQHVPEELDLSSSSQMSPLSLYGENSNSLSSPEPLKEDKPVTGSRNKTENGLTPKKKIQVNSKPSIQPKPLLLPAAPKTQTNSSVPAKTIIIQTVPTLMPLAKQQPIISLQPAPTKGQTVLLSQPTVVQLQAPGVLPSAQPVLAVAGGVTQLPNHVVNVVPAPSANSPVNGKLSVTKPVLQSTMRNVGSDIAVLRRQQRMIKNRESACQSRKKKKEYMLGLEARLKAALSENEQLKKENGTLKRQLDEVVSENQRLKVPSPKRRVVCVMIVLAFIILNYGPMSMLEQDSRRMNPSVGPANQRRHLLGFSAKEAQDTSDGIIQKNSYRYDHSVSNDKALMVLTEEPLLYIPPPPCQPLINTTESLRLNHELRGWVHRHEVERTKSRRMTNNQQKTRILQGVVEQGSNSQLMAVQYTETTSSISRNSGSELQVYYASPRSYQDFFEAIRRRGDTFYVVSFRRDHLLLPATTHNKTTRPKMSIVLPAININENVINGQDYEVMMQIDCQVMDTRILHIKSSSVPPYLRDQQRNQTNTFFGSPPAATEATHVVSTIPESLQ

SEQ ID NO: 5 - DNA, 948 bp, Artificial Sequence
  CHO eIF2alpha mutant
atgccgggtctaagttgtagattttatcaacacaaattccctgaggtggaagatgtagtgatggtgaatgtaagatccatagctgagatgggagcctatgttagcttgttggaatacaataacattgaaggcatgattcttcttagtgaattagccagaagacgtatccgttctataaacaaactgatccgcattggcagaaatgaatgtgtagttgtcattagagtggacaaagaaaaaggatatattgatttgtcaaaaagaagagtgtctccagaggaagcgattaaatgtgaagacaaattcacaaaatccaaaactgtttatagcattcttcgacatgttgctgaggtattagaatataccaaggatgaacaactggaaagcctgttccagaggactgcctgggtctttgatgacaagtacaagaaacctggatatggtgcttatgatgcctttaagcatgcagtctcagacccatctatcttggatagtttagatttgaatgaaaatgaacgagaagtactcattaacaatatcaatagacgtttgacaccacaagctgtcaaaattcgagcagatattgaagtagcctgctatggttatgagggaattgatgctgtaaaggaagccctgagagccggtttgaattgttctacagaaactatgcccatcaagattaacctaatagctccacccaggtatgtgatgacaacaactacactagagagaacagaaggcctctctgttctcaatcaggctatggcagtcatcaaagagaagattgaggaaaagaggggtgtgttcaatgttcagatggagcccaaagtggtcacagatacagatgagactgaacttacaaggcagatggaacggctggagagagaaaatgcagaagtggatggagatgatgatacagaagagatggaagccaaagctgaagattaa

SEQ ID NO: 6 - protein, 315 aa, Artificial Sequence
  CHO eIF2alpha mutant
MPGLSCRFYQHKFPEVEDVVMVNVRSIAEMGAYVSLLEYNNIEGMILLSELARRRIRSINKLIRIGRNECVVVIRVDKEKGYIDLSKRRVSPEEAIKCEDKFTKSKTVYSILRHVAEVLEYTKDEQLESLFQRTAWVFDDKYKKPGYGAYDAFKHAVSDPSILDSLDLNENEREVLINNINRRLTPQAVKIRADIEVACYGYEGIDAVKEALRAGLNCSTETMPIKINLIAPPRYVMTTTTLERTEGLSVLNQAMAVIKEKIEEKRGVFNVQMEPKVVTDTDETELTRQMERLERENAEVDGDDDTEEMEAKAED

(19) United States
(12) Patent Application Publication
(10) Pub. No.: US 2005/0106222 A1
(43) Pub. Date: May 19, 2005

(54) Method to increase protein production in culture

(76) Inventors: Eric N. Ailor, San Diego, CA (US); Mitchell E. Reff, San Diego, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/864,348

(22) Filed: Jun. 10, 2004

Related U.S. Application Data
(60) Provisional application No. 60/477,399, filed on Jun. 11, 2003.

(51) Int. Cl. A61K 9/70; B32B 27/12

(52) U.S. Cl. 424/443; 442/123

(57) ABSTRACT

A new recombinant protein production methodology has been discovered for expression of genes of interest. This should be useful for low or poorly expressing genes and in cellular production of high levels of proteins when such high levels may be detrimental or stressful to cells. Isolated polynucleotides encoding a spliced form XBP-1, ATF6, and eIF2α S51A are disclosed. Expression vectors comprising these polynucleotides are useful in increasing the specific cellular productivity in a cell expressing a polypeptide of interest by augmenting the unfolded protein response in a cell. Finally, methods to increase specific cellular productivity in mammalian cells using the above mentioned polynucleotides are also disclosed.

This application claims the benefit of U.S. Provisional Application No. 60/477,399 filed Jun. 11, 2003, which is herein incorporated by reference.

### BACKGROUND OF THE INVENTION

1. Field of Invention

The invention relates to isolated polynucleotides encoding the spliced form of the transcription factor XBP-1 and selectable markers, vectors containing one or more of the isolated polynucleotides, and methods of using the vectors to increase the specific cellular productivity of a given cell.

2. Background

The generation of functional secreted proteins requires efficient and undisturbed functioning of the endoplasmic reticulum (ER)-Golgi secretory pathway in cells (Cudna and Dickson, Biotechnol. & Bioeng. 81:56-65 (2002). This secretory pathway can become congested with unfolded or misfolded proteins during production of large quantities of recombinant proteins.

The need to overcome cellular stress causes cells to activate a variety of mechanisms. In one such pathway, the alpha subunit of polypeptide chain initiation factor eIF2 is phosphorylated by a number of related protein kinases. (Clemens M J, Prog. Mol. Subcell. Biol. 27:57-89 (2001)). Phosphorylated eIF2α acts as a dominant inhibitor of the guanine nucleotide exchange factor eIF2β and prevents the recycling of eIF2β between successive rounds of protein synthesis. Extensive phosphorylation of eIF2α and strong inhibition of eIF2β activity can result in the downregulation of the overall rate of protein synthesis. These mechanisms provide a signal transduction pathway linking eukaryotic cellular stress responses to alterations in the control of gene expression at the translational level.

In addition, the ER also plays an important role as a signaling compartment and a sensor of cellular stress. The ER is a principal site for folding and maturation of transmembrane, secretory, and ER-resident proteins (Liu and Kaufman, J. Cell Sci. 116:1861-1862 (2003)). Internal and external factors that alter ER homeostasis can lead to the accumulation of unfolded proteins, which are a threat to all living cells. In response to stress, eukaryotic cells activate an intracellular signaling pathway known as the unfolded protein response (UPR). Cellular stresses can be both physical and chemical in nature, and may include heat shock, nutrient deprivation, changes in intracellular calcium, changes in pH, changes in dissolved O₂, accumulation of unfolded or denatured proteins, the induction of apoptosis, and any other stress that upregulates the UPR.

The UPR is mediated by a multifaceted intracellular signaling pathway triggered by inhibition of glycosylation, Ca⁺² depletion, and other stress conditions that interfere with protein folding in the ER (Gass et al., J. Biol. Chem. 277:49047-49054 (2002)). As unfolded proteins accumulate, the UPR coordinates a broad downregulation of protein synthesis with increased expression of various gene products including ER resident molecular chaperones that promote protein folding and secretion. The UPR coordinates this intracellular signaling pathway by transcriptional induction of UPR genes, translational attenuation of global protein synthesis, and ER-associated degradation (ERAD) via the proteosome (Liu and Kaufman, J. Cell Sci. 116:1861-1862 (2003)). These diverging pathways provide adaptive responses for survival. If the protein accumulation defect is not corrected, cells will undergo apoptosis.

When the mammalian ER is stressed, a set of ER transmembrane proteins initiates the UPR. One of these is ATF6α, a 90 kDa type II transmembrane protein that undergoes ER stress-induced proteolysis to liberate its 50 kDa cytosolic domain (p50ATF6α), which is a basic leucine zipper transcription factor. p50ATF6α translocates to the nucleus and participates in transcriptional induction of genes including BiP and GRP94, two ER resident molecular chaperones; XBP-1 (X-box binding protein 1), a basic leucine zipper transcription factor; and CHOP (C/EBP homologous protein, also known as GADD153, a member of the CAAT/enhancer-binding protein family of transcription factors (Gass et al., J. Biol. Chem. 277:49047-49054 (2002); Ma and Hendershot, Cell 107:827-830 (2001)).

In parallel with ATF6, ER stress-inducing agents activate the IRE1α/β proteins. IRE1α is a ubiquitously expressed ER type I transmembrane protein containing both a serine/threonine kinase module and an endoribonuclease domain in its cytosolic region. Upon UPR activation, IRE1α executes site-specific cleavage of ABP-1 mRNA to remove a 26-nucleotide intron. Religation of the 5′ and 3′ fragments yields a spliced ABP-1 mRNA with an altered reading frame encoding a 54 kDa basic leucine zipper transcription factor. p54XBP-1 is more potent as a transcriptional activator and is likely more stable than the 30 kDa protein translated from unprocessed XBP-1 mRNA. (Gass et al., J. Biol. Chem. 277:49047-49054 (2002); Reimold et al., Nature 412:300-307 (2001)). Therefore, it appears that ATF6 and IRE1α work together to regulate expression of XBP-1, ultimately generating a transcriptional activator that amplifies the UPR.

ER stress also mediates a death-signaling pathway by transcriptional activation of genes encoding proapoptotic functions. Activation of UPR sensor IRE1, PERK, or ATF6 leads to transcriptional activation of CHOP/GADD153, a bZIP transcription factor that potentiates apoptosis.

There is a need in the art for methods to increase yields of recombinantly produced proteins in eukaryotic systems. This invention meets that need, as well as others, by increasing yields of polypeptides of interest.

### BRIEF SUMMARY OF THE INVENTION

A new recombinant protein production methodology has been discovered for expression of genes of interest. This should be useful for many different applications, including but not limited to, low or poorly expressing genes and in cellular production of high levels of proteins when such high levels may be detrimental or stressful to cells. As a result of this invention, increases in production quantities of proteins can be obtained from eukaryotic cells.

One embodiment of the invention is directed to a method of increasing specific cellular productivity of a membrane or secreted polypeptide of interest in a cell, comprising expressing the polypeptide of interest, wherein the cell also expresses an exogenous, spliced form of XBP-1. In another embodiment, the polynucleotide encoding a polypeptide is introduced into the cell expressing an exogenous, spliced form of XBP-1. In yet another embodiment, the polynucleotide encoding the polypeptide of interest is exogenous.

In some embodiments, the claimed method may “consist essentially” of the steps described in the method.

In additional embodiments, the method results in at least about a 5% increase in specific cellular productivity of the polypeptide of interest relative to control cells transfected with the polypeptide of interest without XBP-1.

In various embodiments of the invention, the method results in increases in the specific cellular productivity of a polypeptide relative to control cells previously transfected with the polypeptide of interest without XBP-1, wherein the increases are about 5% to about 10%, about 11% to about 20%, about 21% to about 30%, about 31% to about 40%, about 41% to about 50%, about 51% to about 60%, about 61% to about 70%, about 71% to about 80%, about 81% to about 90%, or about 91% to about 100%.

In another embodiment of the invention, cells of the method further comprise a selectable marker. The selectable marker may be used to detect cells which comprise the polypeptide of interest. In one embodiment of the invention, the cells comprising the polypeptide of interest are detected by selecting for stable integration of the selectable marker.

In one embodiment of the invention, the method results in the production of the polypeptide of interest at a level of at least about 5 pg/cell/day. In another embodiment, the polypeptide of interest is Hu24-31.1.

In another embodiment of the invention, the spliced form of XBP-1 is encoded by a polynucleotide which encodes the polypeptide of SEQ ID NO:2 or by the polynucleotide of SEQ ID NO:1. In yet another embodiment, the spliced form of XBP-1 is a derivative of an XBP-1 splice variant that has XBP-1 activity.

The invention is also directed to a method of increasing specific cellular productivity of a polypeptide of interest wherein the above-described vector further contains additional polynucleotides. In one embodiment of the invention, the additional polynucleotides encode a transcription factor or a translation enhancer. In another embodiment of the invention, the additionally encoded transcription factor is ATF6 or its activation domain. In a further embodiment, ATF6 is encoded by a polynucleotide encoding the polypeptide of SEQ ID NO:4. In yet another embodiment, ATF6 is encoded by a derivative of SEQ ID NO:4, whereby the polypeptide retains ATF6 activity. In another embodiment of the invention, the translation enhancer is eIF2α S51A. In a further embodiment, eIF2α S51A is encoded by a polynucleotide encoding the polypeptide of SEQ ID NO:6. In yet another embodiment, eIF2α S51A is encoded by a derivative of SEQ ID NO:6, whereby the polypeptide retains eIF2α S51 A activity.

The polypeptide of interest can be any polypeptide that is desired to be produced in large quantities. In one embodiment of the invention, the polypeptide of interest is an antibody or antibody fragment. The antibody can be monoclonal, polyclonal, mammalian, murine, chimeric, humanized, human, primate, or primatized. In one embodiment, the antibody is a monoclonal antibody. In a further embodiment, the antibody is chimeric. The antibody fragments generally include immunoglobulin light chain, immunoglobulin heavy chain, immunoglobulin light and heavy chains, Fab, F(ab′)₂, Fc, Fc-Fc fusion proteins, Fv, single-chain Fv, tetravalent single chain Fv, disulfide-linked Fv fragments, domain deleted, minibody, and diabody. In another embodiment of the invention, the polypeptide of interest is a C_{H}2 domain deleted antibody.

In one embodiment of the invention, the method can be used to make an antibody or antibody fragment. In a further embodiment, the antibody is the anti-CD 154 antibody Hu24-31.1.

Specific cellular productivity can be increased in a large variety of cells. Therefore, the invention is related to methods of increasing specific cellular production in eukaryotic, insect, and mammalian cells. In one embodiment of the invention the mammalian cells may be monkey kidney CV1, monkey kidney COS, human lens epithelium PER.C6™, human embryonic kidney, baby hamster kidney, african green monkey kidney, human cervical carcinoma, canine kidney, buffalo rat liver, human lung, human liver, mouse mammary tumor cells, and myeloma cells. In one embodiment, the myeloma cells can be NS0, Sp2/0, or Ag8653. In a further embodiment of the invention, the mammalian cells are chinese hamster ovary cells.

The invention is also directed to a method of increasing specific cellular productivity of a polypeptide of interest in a cell, wherein the method comprises introducing into a cell a vector comprising two polynucleotides, wherein the first polynucleotide encodes the spliced form of the transcription factor XBP-1 and the second polynucleotide encodes a polypeptide of interest, and expressing the polypeptide of interest.

The invention further relates to an expression vector for increasing specific cellular productivity of a membrane or secreted polypeptide of interest comprising a first polynucleotide and a second polynucleotide, wherein the first polynucleotide comprises a polynucleotide which encodes a spliced form of the transcription factor XBP-1 and the second polynucleotide comprises a polynucleotide which encodes a selectable marker. In one embodiment of the invention, the XBP-1 polypeptide is encoded by the polynucleotide of SEQ ID NO:1. In a further embodiment of the invention, the XBP-1 polypeptide is encoded by a polynucleotide which is 95% identical to SEQ ID NO:1, wherein the 95% identical polynucleotides produce an increased specific cellular productivity when introduced into a cell expressing a polypeptide of interest.

The invention further relates to an expression vector wherein the polynucleotides which increase specific cellular productivity comprises nucleotides 195 to 356 of SEQ ID NO:1, wherein the nucleotides 195 to 356 produce an increased specific cellular productivity when introduced into a cell expressing a polypeptide of interest.

In one embodiment, the first polynucleotide of the expression vector encodes the polypeptide of SEQ ID NO:2.

The invention further relates to the above described expression vector wherein the vector further comprises additional polynucleotides. In one embodiment of the invention, the additional polynucleotides encode either a second transcription factor or a translation enhancer. In another embodiment, the second transcription factor is ATF6 or its activation domain. In a further embodiment of the invention, ATF6 is encoded by a polynucleotide encoding the polypeptide of SEQ ID NO:4. In another embodiment of the invention, the translation enhancer is eIF2α S51A. In a further embodiment, the eIF2α S51A is encoded by a polynucleotide encoding the polypeptide of SEQ ID NO:6.

The invention further relates to a cell comprising the above described expression vector. In a particular embodiment, the cell is an animal cell. In one embodiment, the cell is an eukaryotic cell. In another embodiment, the cell is an insect cell. In yet another embodiment, the cell is a mammalian cell. In a further embodiment, the mammalian cell can a monkey kidney CV1, monkey kidney COS, human lens epithelium PER.C6™, human embryonic kidney, baby hamster kidney, african green monkey kidney, human cervical carcinoma, canine kidney, buffalo rat liver, human lung, human liver, mouse mammary tumor cell, or myeloma cell line. In one embodiment, the myeloma cells may be NS0, Sp2/0, or Ag8653. In yet another embodiment of the invention, the cell is a chinese hamster ovary cell.

The invention further relates to an expression vector which comprises a polynucleotide which upregulates the unfolded protein response in a cell.

The invention further relates to an isolated polynucleotide comprising a first component encoding the spliced form of XBP-1 and a second component encoding a selectable marker, wherein the isolated polynucleotide, when introduced into a cell expressing a polypeptide of interest, produces an increased specific cellular productivity of a polypeptide of interest of at least 5 pg/cell/day in a cell.

In another embodiment of the invention, the isolated polynucleotide comprises a polynucleotide encoding a polypeptide of interest.

In one embodiment, the isolated polynucleotide comprises a spliced form of XBP-1 encoded by a polynucleotide encoding the polypeptide of SEQ ID NO:2.

In another embodiment of the invention, the isolated polynucleotide further comprises one or more additional polynucleotides. In one embodiment, the additional polynucleotides encode a transcription factor or a translation enhancer. In another embodiment, the transcription factor is ATF6 or its activation domain. In a further embodiment, the ATF6 is encoded by the polynucleotide of SEQ ID NO:3, or by encoding the polypeptide of SEQ ID NO:4. In another embodiment, the translation enhancer is eIF2α S51A. In a further embodiment, eIF2α S51A is encoded by the polynucleotide of SEQ ID NO:5, or by encoding the polypeptide of SEQ ID NO:6.

The invention further relates to a method of increasing specific cellular productivity of a membrane or secreted polypeptide of interest in a cell, the method comprising introducing into a cell expressing a polypeptide of interest, an isolated polynucleotide comprising a first component encoding the spliced form of XBP-1 and a second component encoding a selectable marker, wherein the isolated polynucleotide produces an increased specific cellular productivity of the polypeptide of interest of at least 5 pg/cell/day in the cell.

### BRIEF DESCRIPTION OF THE FIGURES

FIGS. 1A (SEQ ID NO:1) and 1B (SEQ ID NO:2) are the nucleotide and spliced protein sequences of XBP-1 from chinese hamster ovary cells, respectively. FIG. 1B shows the predicted DNA binding domain underlined, and the predicted transactivation domain is shown in capital letters.

FIGS. 2A (SEQ ID NO:3) and 2B (SEQ ID NO:4) are the nucleotide and protein sequences of human ATF6, respectively. FIGS. 2A and 2B show nuclear ATF6 in bold. In FIG. 2B, the predicted transactivation domain is shown capital letters, the predicted DNA binding domain is shown by solid underline, the predicted transmembrane domain is boxed, and the ER domain is shown by dotted underline.

FIGS. 3A (SEQ ID NO:5) and 3B (SEQ ID NO:6) are the nucleotide and protein sequences of the eIF2α S51A mutant from CHO cells, respectively. FIG. 3A shows substitution of a thymine for guanine residue (bolded underline), resulting in a serine to alanine mutation at amino acid position 51 in FIG. 3B (bolded underline).

FIG. 4 shows a cell line screening using cells grown in spinner flasks. Specific productivity (PCD) and doubling time of several S8 cell lines compared to 500E9 is shown. Data is compiled from a minimum of three spinner flasks cultured for 3 to 4 days. Values on the y-axis for doubling time are reported in hours and values for PCD are reported as pg protein/cell/day.

FIG. 5 shows a cell line screening using cells grown in T-flasks. Specific productivity (PCD) and doubling time of several cell lines compared to 500E9 after 5 days residence in a single T-75 tissue culture flask is shown. Values on the y-axis for doubling time are reported in hours and values for PCD are reported as pg protein/cell/day.

FIG. 6 shows the evaluation of final candidate cell lines. Specific productivity (PCD's) and doubling time of final cell line candidates compared to 500E9 is shown. Spinner flasks were set up in triplicate. The PCD and doubling time values were averaged from days 2 to 4 of the cultures. Values on the y-axis for doubling time are reported in hours and values for PCD are reported as pg protein/cell/day.

### DETAILED DESCRIPTION OF THE INVENTION

The invention produces increases in the specific cellular productivity of a particular polypeptide of interest. Without being constrained to a proposed mechanism, it is believed that the increase in specific cellular productivity results from an upregulation of the cell's unfolded protein response resulting from expression of a spliced form of XBP-1 protein to levels greater than that found in most mammalian cells. This information can be utilized to increase the levels of production of polypeptides, especially those that are difficult to express.

“Exogenous” means originating or produced outside the cell, body, or organ.

“Specific cellular productivity” means the level of protein synthesis or amount of polypeptide secreted from a given number of cells over a specified period of time. Values for specific cellular productivity are generally reported as pg protein/cell/day.

As used herein “transcription factor” means a polypeptide required for altering recognition by RNA polymerases of specific modulatory sequences in eukaryotic genes (e.g. stimulating or down regulating sequences). Common structural features of transcription factors include metal-binding domains known as zinc fingers, and basic residues encompassing leucine zippers.

“About” means plus or minus 5% of the value in question. Therefore, for example, “about 20%” means 19% to 21%.

“A,” “an,” “the,” and the like, unless otherwise indicated, may include plural forms. Also, reference to singular forms also embrace plural forms. Therefore, for example, polynucleotide also encompasses polynucleotides.

XBP-L

The XBP-1 gene has two open reading frames (ORFs) which encode both an unspliced and spliced version of the polypeptide. The term “gene” refers to a nucleic acid whose nucleotide sequence encodes an RNA or polypeptide. A gene can be either RNA or DNA. Genes may include regions preceding and following the coding region (leader and trailer) as well as intervening sequences (introns) between individual coding segments (exons).

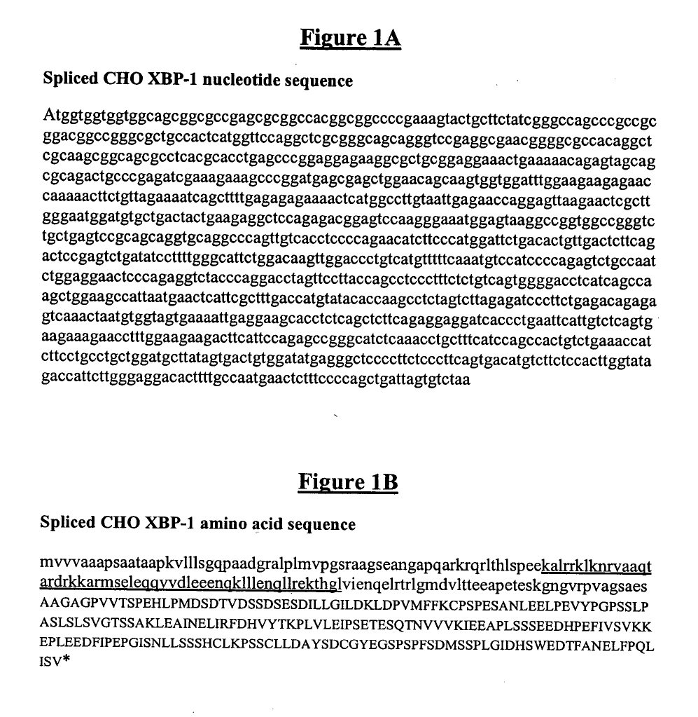

XBP-1 mRNA is spliced in response to ER stress. ER stress can be caused by a variety of mechanisms including the accumulation of unfolded/misfolded proteins within the lumen of the ER. Unspliced XBP-1 mRNA expressed at a low level in unstressed mammalian cells is translated, while under conditions of ER stress, the XBP-1 intron is excised. The spliced XBP-1 gene product has an amino acid sequence of about 370 amino acid residues whose initiation codon is at position 1-3 of the nucleotide sequence shown in FIG. 1A (SEQ ID NO:1), with a deduced molecular weight of about 54 kDa. The amino acid sequence of the spliced XBP-1 protein from Chinese Hamster Ovary (CHO) cells is shown in FIG. 1B (SEQ ID NO:2). The mature XBP-1 protein has two main structural domains. The first major structural region is the basic leucine zipper region, which includes the DNA binding domain, and is predicted to correspond to amino acid residues from about 65 to about 118 in FIG. 1B. The other major structural region is the transactivation domain, which has been predicted to correspond to amino acid residues from about 160 to about 370 in FIG. 1B. It will be appreciated that persons of skill in the art may disagree, depending on the criteria used, concerning the exact ‘address’ of the above described XBP-1 protein domains. Thus, for example, the exact location of the XBP-1 protein DNA binding and transactivation domains in FIG. 1B (SEQ ID NO:2) may vary slightly (e.g., the exact ‘address’ may differ by about 1 to about 5 residues compared to that shown in FIG. 1B) depending on the criteria used to define the domain. Generally, this is defined as the regions which bind or transactivate such that said binding or transactivating results in increased specific cellular productivity of the polypeptide of interest.

In one embodiment, the invention relates to any derivatives of the spliced variants of XBP-1 which retain XBP-1 activity. Such derivatives include those produced by nucleotide substitutions, deletions or additions, which may involve one or more nucleotides. Alterations in the XBP-1 coding regions may produce conservative or non-conservative amino acid substitutions, deletions or additions. In one embodiment, the alterations are silent substitutions, additions and deletions, which do not alter the properties and activities of the XBP-1 protein or portions thereof. In another embodiment, the alterations are conservative substitutions.

“Polynucleotide” or “nucleic acid molecule”, as used interchangeably herein, refers to nucleotide polymers of any length, such as two or more, and includes both DNA and RNA. The nucleotides can be deoxyribonucleotides, ribonucleotides, nucleotide analogs (including modified phosphate moieties, bases, or sugars), or any substrate that can be incorporated into a polymer by a suitable enzyme, such as a DNA polymerase or an RNA polymerase. Thus, a polynucleotide may comprise modified nucleotides, such as methylated nucleotides, and their analogs. Further, any of the hydroxyl groups ordinarily present on the pentose (i.e., ribose or deoxyribose) ring of a nucleotide may be, for example, replaced by phosphonate or phosphate groups, protected by standard protecting groups, activated to prepare additional linkages to additional nucleotides, or conjugated to a solid support. The 5′ and 3′ terminal OH groups on the pentose ring of a nucleotide can be phosphorylated or substituted with amines or organic capping group moieties of from about 1 to about 50 carbon atoms. Other hydroxyl groups on the ribose or deoxyribose ring may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, anomeric sugars, epimeric sugars, such as arabinose, xylose, pyranose sugars, furanose sugars, sedoheptuloses, acyclic analogs, and a basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl, or araldyl. Not all linkages in a polynucleotide need be identical. The preceding description applies to all polynucleotides referred to herein, including RNA and DNA.

“Nucleotide” or “NTP” refers to a base-sugar-phosphate compound. “Base” refers to a nitrogen-containing ring molecule that, when combined with a pentose sugar and a phosphate group, form a nucleotide. Bases include single ring pyrimidines, such as cytosine (C), thymine (T), and uracil (U), and double ring purines, such as adenine (A) and guanine (G). “Sugar” or “pentose sugar” generally refers to a pentose ring, such as a ribose ring or deoxyribose ring. Nucleotides are the monomeric subunits of both types of nucleic acid polymers, that is, RNA and DNA. “Nucleotide” or “NTP” refers to any nucleoside 5′ phosphate, that is, ribonucleoside 5′ phosphates (i.e., mono-, di-, and triphosphates) and deoxyribonucleoside 5′ phosphates (i.e., mono-, di-, and triphosphates), and includes “nucleoside phosphate analogs”, “nucleotide analogs”, and “NTP analogs”. “Nucleoside phosphate analog”, “nucleotide analog”, and “NTP analog” refer to any nucleoside 5′ phosphate (i.e., mono-, di-, or triphosphate) which is analogous to a native nucleotide but which contains one or more chemical modifications when compared to the corresponding native nucleotide. Nucleotide analogs include base-modified analogs (e.g. 5-mercapto pyrimidines, 8-mercapto purines), phosphate-modified analogs (e.g., α-thio-triphosphates), and sugar-modified analogs (3′ OMe, 3′deoxy) and may comprise modified forms of deoxyribonucleotides as well as ribonucleotides.

Unless otherwise indicated, each “polynucleotide sequence” set forth herein is presented as a sequence of deoxyribonucleotides (abbreviated A, G, C and T). However, by “polynucleotide sequence” for an RNA molecule or polynucleotide, the corresponding sequence of ribonucleotides (A, G, C and U) may be replaced where each thymidine deoxynucleotide (T) in the specified deoxynucleotide sequence with the ribonucleotide uridine (U). For instance, reference to a RNA molecule having the sequence of SEQ ID NO:1 set forth using deoxyribonucleotide abbreviations is intended to indicate an RNA molecule having a sequence in which each deoxynucleotide A, G or C of SEQ ID NO:1 has been replaced by the corresponding ribonucleotide A, G or C, and each deoxynucleotide T has been replaced by a ribonucleotide U.

Thus, in one aspect, isolated polynucleotides are provided which encode the XBP-1 protein, or a splice variant thereof, as further described herein. Further provided are polynucleotides which encode a selectable marker, which may or may not be dominant. As used herein, a “selectable marker” is a gene sequence or protein encoded by that gene sequence; expression of the protein encoded by the selectable marker assures that a host cell transfected with an isolated polynucleotide which includes the selectable marker will survive a selection process which would otherwise kill a host cell not containing this protein.

By “isolated” polynucleotides is intended a nucleic acid molecule, DNA or RNA, which has been removed from its native environment. For example, a polynucleotide or a polypeptide naturally present in a living animal is not “isolated,” but the same polynucleotide or polypeptide separated from the coexisting materials of its natural state is “isolated”, as the term is employed herein. Therefore, recombinant DNA molecules contained in a vector are considered isolated for the purposes of the invention. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith and Johnson, Gene 67:31-40 (1988). The terms isolated and purified are sometimes used interchangeably.

By “isolated” is meant that the DNA is free of the coding sequences of those genes that, in the naturally-occurring genome of the organism (if any) from which the DNA of the invention is derived, immediately flank the gene encoding the DNA of the invention. The isolated DNA may be single-stranded or double-stranded, and may be genomic DNA, cDNA, recombinant hybrid DNA, or synthetic DNA. It may be identical to native DNA sequence, or may differ from such sequence by the deletion, addition, or substitution or one or more nucleotides.

“Isolated” or “purified” as it refers to preparations made from biological cells or hosts should be understood to mean any cell extract containing the indicated nucleic acid or protein including a crude extract of the polynucleotide or polypeptide of interest. Furthermore, a preparation of nucleic acid or protein that is “pure” or “isolated” should be understood to mean a preparation free from naturally occurring materials with which such nucleic acid or protein is normally associated in nature.

Further examples of isolated DNA molecules include recombinant DNA molecules maintained in heterologous host cells or purified (partially or substantially) DNA molecules in solution. Isolated RNA molecules include in vitro RNA transcripts of the DNA molecules of the invention. Isolated nucleic acid molecules according to the invention further include such molecules produced synthetically.

Isolated polynucleotides of the invention include DNA molecules comprising an ORF whose initiation codon is at position 1-3 of the nucleotide sequence shown in FIG. 1A (SEQ ID NO:1) and further include DNA molecules which comprise a sequence substantially different than all or part of the ORF whose initiation codon is at position 1-3 of the nucleotide sequence shown in FIG. 1A (SEQ ID NO:1) but which, due to the degeneracy of the genetic code, still encode the spliced XBP-1 protein or a functional fragment thereof. By “functional fragment” is meant a fragment of XBP-1 that when expressed in a cell results in the increased specific cellular productivity of a polypeptide of interest. In addition, the isolated polynucleotides include a polynucleotide sequence which encodes a polypeptide of interest. Of course, the genetic code is well known in the art. Thus, it would be routine for one skilled in the art to generate the degenerate variants described above.

Isolated polynucleotides of the invention also include polynucleotides encoding: a polypeptide comprising the XBP-1 protein DNA binding domain (amino acid residues from about 65 to about 118 in SEQ ID NO:2); a polypeptide comprising the XBP-1 protein transactivation domain (amino acid residues from about 160 to about 370 in SEQ ID NO:2); and a polypeptide comprising the XBP-1 protein DNA binding and transactivation domains (amino acid residues from about 65 to about 370 in SEQ ID NO:2).

Further embodiments of the invention include isolated nucleic acid molecules comprising a polynucleotide having a nucleotide sequence at least 95% identical, and at least 96%, 97%, 98% or 99% identical to (a) a nucleotide sequence encoding the polypeptide having the amino acid sequence in SEQ ID NO:2; (b) a nucleotide sequence encoding the XBP-1 DNA binding domain; and (c) a nucleotide sequence complementary to any of the nucleotide sequences in (a) and (b).

By a polynucleotide having a nucleotide sequence at least, for example, 95% “identical” to a reference nucleotide sequence encoding a XBP-1 polypeptide is intended that the nucleotide sequence of the polynucleotide is identical to the reference sequence except that the polynucleotide sequence may include up to five point mutations per each 100 nucleotides of the reference nucleotide sequence encoding the XBP-1 polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. The 95% identical sequences have XBP-1 activity as defined by the ability to result in the increased specific cellular productivity of at least 5% of a polypeptide of interest when expressed in a cell.

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith D. W., ed., Academic Press, New York, 1993; Computer analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

As a practical matter, whether any particular nucleic acid molecule has a nucleotide sequence at least 95%, 96%, 97%, 98% or 99% identical to, for instance, the nucleotide sequence shown in FIG. 1A can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman (Advances in Applied Mathematics 2:482-489 (1981)) to find the best segment of homology between two sequences. When using Bestfit or any other sequence alignment program to determine whether a particular sequence is, for instance, 95% identical to a reference sequence according to the invention, the parameters are set, of course, such that the percentage of identity is calculated over the full length of the reference nucleotide sequence and that gaps in homology of up to 5% of the total number of nucleotides in the reference sequence are allowed.

Other computer program methods to determine identity and similarity between two sequences include, but are not limited to GCG program package (Devereux, J., et al., Nucleic Acids Research 12(I):387 (1984)), BLASTP, BLASTN, FASTA (Atschul, S. F. et al., J. Mol. Biol. 215:403 (1990)).

Embodiments of the invention may include nucleic acid molecules which are at least 95%, 96%, 97%, 98% or 99% identical to the nucleic acid sequence shown in FIG. 1A which encode a polypeptide having XBP-1 protein activity. By “a polypeptide having XBP-1 protein activity” is intended polypeptides exhibiting similar, but not necessarily identical, activity as compared to the XBP-1 protein as measured in a particular biological assay.

Due to the degeneracy of the genetic code, one of ordinary skill in the art will recognize that large numbers of the polynucleotides at least 95%, 96%, 97%, 98% or 99% identical to the nucleic acid sequence shown in FIG. 1A (SEQ ID NO:1) will encode a polypeptide “having XBP-1 protein activity.” In fact, since degenerate variants all encode the same polypeptide, this will be clear to the skilled artisan even without performing the above described comparison assay. It will be further recognized in the art that, for such polynucleotides that are not degenerate variants, a reasonable number will also encode a polypeptide having XBP-1 protein activity. This is because the skilled artisan is fully aware of amino acid substitutions that are either less likely or not likely to significantly effect protein function (e.g., replacing one aliphatic amino acid with a second aliphatic amino acid). As used herein a “substitution” results from the replacement of one or more amino acids or nucleotides in a polypeptide or nucleic acid, respectively.

For example, guidance concerning how to make phenotypically silent amino acid substitutions is provided in Bowie, J. U., et al., “Deciphering the Message in Protein Sequences: Tolerance to Amino Acid Substitutions,” Science 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie et al., supra, and the references cited therein.

As indicated, changes are generally of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

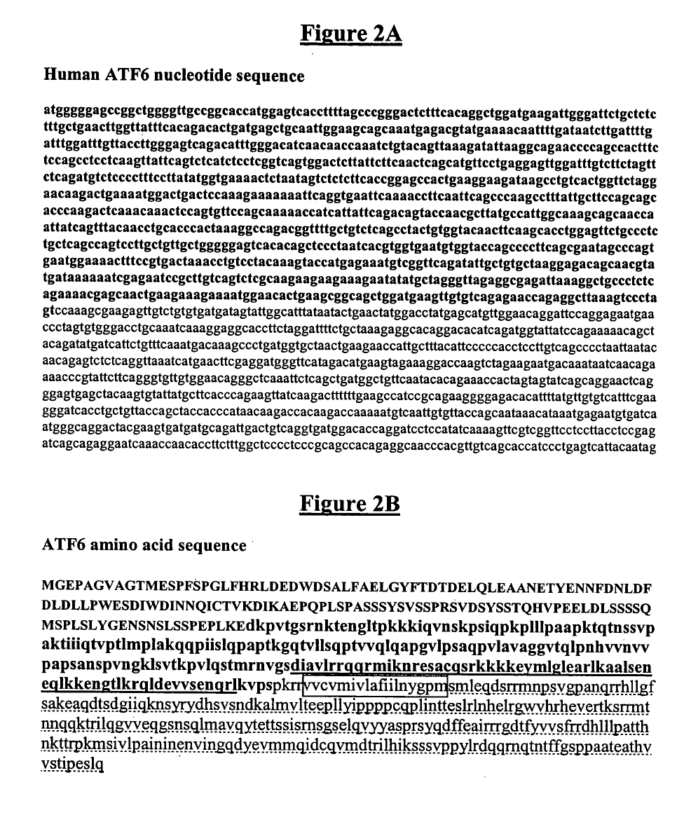

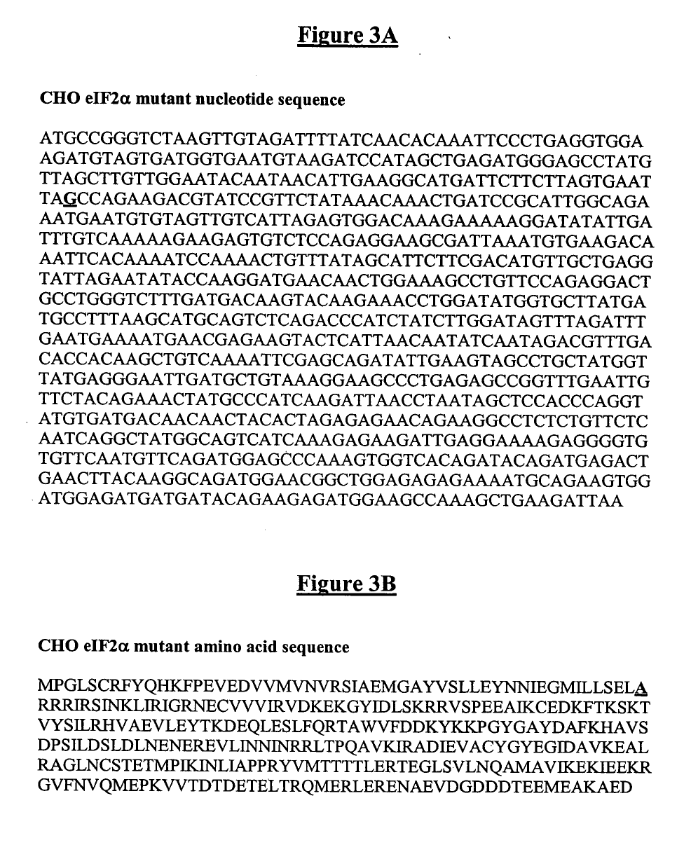

In a further embodiment of the invention, the isolated XBP-1 polynucleotides further comprise additional polynucleotides. These additional polynucleotides can also be polynucleotides that encode polypeptides of the unfolded protein response pathway. In one embodiment of the invention, the additional polynucleotides encode a second transcription factor of the unfolded protein response pathway. In another embodiment of the invention, the XBP-1 polynucleotides further comprise polynucleotides that encode the ATF6 polypeptide or its activation domain (FIG. 2A-2B) (SEQ ID NOs:3-4), or a functional fragment thereof. By “functional fragment” is meant a fragment of ATF6 that when expressed in a cell expressing XBP-1, results in the increased specific cellular productivity of a polypeptide of interest. In another embodiment of the invention, the additional polynucleotides encode a translation enhancer. In a further embodiment of the invention, the translation enhancer is eIF2α S51A (FIG. 3A-3B) (SEQ ID NOs:5-6), or a functional fragment thereof. By “functional fragment” is meant a fragment of eIF2α S51A that when expressed in a cell expressing XBP-1, results in the increased specific cellular productivity of a polypeptide of interest.

Polypeptides of Interest

As used herein, “polypeptide of interest” refers generally to peptides and proteins having more than about ten amino acids. The polypeptides may be endogenous to the host cell, or may be exogenous, meaning that they are heterologous, i.e., foreign, to the host cell being utilized, such as a human protein produced by a CHO cell, or a yeast polypeptide produced by a mammalian cell. In one embodiment, mammalian polypeptides (polypeptides that were originally derived from a mammalian organism) are used, such as those which are directly secreted into the medium, or can be made to be secreted, or engineered, by means known to one of skill in the art, to be secreted.

Examples of mammalian polypeptides of interest include, but are not limited to molecules such as renin, a growth hormone, including human growth hormone; bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-.beta.; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD2, CD3, CD4, CD8, CD16, CD19, CD20, CD22, CD23, CD32, CD45, CD80, CD86, CDw128, CD154, CD183, CD184, and CD195; erythropoietin; osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-12; superoxide dismutase; T-cell receptors; surface membrane proteins; decay accelerating factor; viral antigen such as, for example, a portion of the AIDS⁻ envelope; transport proteins; homing receptors; addressins; regulatory proteins; antibodies; and fragments of any of the above-listed polypeptides.

In one embodiment of the invention, the polypeptide of interest is an antibody or an antibody fragment.

The term “antibody” as used herein refers to immunoglobulin molecules and immunologically active portions of immunoglobulin (Ig) molecules. Such antibodies may include, but are not limited to, polyclonal, monoclonal, polyclonal, mammalian, murine, human, primate, primatized, humanized, chimeric, single chain, Fab, Fab′ and Fab′₂ fragments, Fc, Fc-Fc fusion proteins, Fv, single chain Fv, tetravalent Fv, domain deleted, immunoglobulin light chain, immunoglobulin heavy chain, immunoglobulin heavy and light chains, minibodies, diabodies and an Fab expression library, or any other form of antibody that would be known to one of skill in the art. Examples can be found in Fundamental Immunology, Paul, W. E. ed., Raven Press, New York, N.Y. (1993). In general, a human antibody molecule relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of antibody species.

It has been shown that fragments of an antibody can perform the function of binding antigens, though it is not necessary that antibody fragments retain the ability to bind antigen. As used herein “antibody fragments” include, but are not limited to: (1) the Fab fragment consisting of V_{L}, V_{H}, C_{L} and C_{H}1 domains; (2) the Fd fragment consisting of the V_{H} and C_{H}1 domains; (3) the Fv fragment consisting of the V_{L} and V_{H} domains of a single antibody; (4) the dAb fragment (Ward, E. S. et al., Nature 341:544-546 (1989)) which consists of a V_{H} domain; (5) isolated CDR regions; (6) F(ab′)₂ fragments; (7) single chain Fv molecules (scFv), wherein a V_{H} domain and a V_{L} domain are linked by a peptide linker which allows the two domains to associate to form an antigen binding site (Bird, et al., Science 242:423-426 (1988); Huston et al., Proc. Natl. Acad. Sci. USA 85:5879-5883 (1988)); (8) tetravalent single chain Fv; (9) bispecific single chain Fv dimers (PCT/US92/09965); (10) domain deleted antibodies; (11) C_{H}2 domain deleted; (12) minibodies; (13) diabodies, multivalent or multispecific fragments constructed by gene fusion (WO 94/13804; P. Holliger et al., Proc. Natl. Acad. Sci. USA 90:6444-6448 (1993); (14) Fc fragments; (15) Fc-Fc fusion proteins (WO 02/088317); (16) antibody light chains; (17) antibody heavy chains; (18) combinations of antibody heavy and light chains.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab)₂ fragment that has two antigen-combining sites and is still capable of cross-linking antigen.

“Fc-Fc fusion protein” is a fusion of two or more polypeptides comprising antibody Fc fragments of the same or different antibody subclass. The Fc fragments can be arranged in any orientation, and may be linked through a spacer molecule, or directly connected. Fc-Fc fusion proteins are useful in crosslinking two cell surface receptors simultaneously. An example of a Fc-Fc fusion protein is described in WO 02/088317 which is incorporated herein by reference.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy chain C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

As used herein the term “domain deleted antibody” shall be held to mean any antibody, or antigen binding fragment or recombinant thereof, in which at least a fraction of one or more of the constant region domains has been deleted or otherwise altered so as to provide desired biochemical characteristics such as increased tumor localization or reduced serum half-life when compared with a whole, unaltered antibody of approximately the same binding specificity. In an embodiment of the invention, one entire domain of the constant region of the modified antibody will be deleted and in another embodiment, the entire C_{H}2 domain will be deleted.

“Single-chain Fv” or “sFv” antibody fragments comprise the V_{H} and V_{L} domains of antibody, wherein these domains are present in a single polypeptide chain. The Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994). Tetravalent single-chain Fv are polypeptides having multiple antigen binding specificities.

“Minibodies” are engineered antibody constructs comprised of the V_{H} and V_{L} domains of a native antibody fused to the hinge region and to the C_{H}3 domain of the immunoglobulin molecule. Minibodies are thus small versions of whole antibodies encoded in a single protein chain which retain the antigen binding region, the C_{H}3 domain to permit assembly into a bivalent molecule and the antibody hinge to accommodate dimerization by disulfide linkages. In contrast, native antibodies are comprised of four chains, two heavy and two light.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

Vectors and Host Cells

The invention also relates to vectors containing the isolated nucleic acid molecules of the invention, host cells containing the recombinant vectors, and the production of XBP-1 polypeptides or fragments thereof, in addition to a polypeptide of interest, by recombinant techniques.

The polynucleotides encoding the XBP-1 and splice variant polypeptides described herein may be incorporated into a vector containing a selectable marker for identification and/or propagation in a host. As discussed in detail below, generally, a plasmid vector may be introduced into a host cell by any suitable method, including as a precipitate, such as a calcium phosphate precipitate, using an electric current, such as electroporation, or in a complex with a charged lipid, and the like. If the vector is a virus, it may be packaged in vitro using an appropriate packaging cell line and then transduced into host cells.

One embodiment for use in the practice of the invention are vectors comprising cis-acting control regions operatively linked to the polynucleotide of interest. Cis-acting control regions include operator and enhancer sequences.

Transcription of the nucleotide sequences encoding the polypeptides of the invention by higher eukaryotes may be increased by inserting an enhancer sequence into the vector. Enhancers are cis-acting elements usually about from 10 to 300 bp that act to increase transcriptional activity of a promoter in a given host cell-type. Examples of enhancers include the SV40 enhancer, which is located on the late side of the replication origin at bp 100 to 270, the cytomegalovirus early promoter enhancer, the polyoma enhancer on the late side of the replication origin, and adenovirus enhancers.

Appropriate trans-acting factors may be supplied by the host, supplied by a complementing vector, or supplied by the vector itself upon introduction into the host.

In certain embodiments in this regard, the vectors provide for specific expression, which may be inducible and/or cell type-specific. Some embodiments include vectors that are inducible by environmental factors that are easy to manipulate, such as temperature and nutrient additives.

Additional expression vectors useful in the invention may include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

The appropriate nucleic acid sequence can be inserted into the vector by a variety of procedures. In general, the nucleic acid sequence is inserted into an appropriate restriction endonuclease site(s) by procedures known in the art. Such procedures and others are deemed to be within the scope of those skilled in the art.

The appropriate polynucleotides will be oriented in the vector in a manner to allow for their expression. Although the specification discusses orientations in which XBP-1 precedes the polypeptide of interest, any orientation which results in expression of the polypeptides of interest is contemplated.

The nucleic acid insert should be operably linked to an appropriate promoter, such as the CMV promoter, the SV40 early and late promoters and promoters of retroviral LTRs, and other promoters known to control expression of genes in eukaryotic cells or their viruses. Other suitable promoters will be known to the skilled artisan. As used herein, the term “promoter” refers to a nucleotide sequence or group of nucleotide sequences which, at a minimum, provides a binding site or initiation site for RNA polymerase action. The expression constructs will further contain sites for transcription initiation, termination and, in the transcribed region, a ribosome binding site for translation. The coding portion of the mature transcripts expressed by the constructs will include a translation initiating at the beginning and a termination codon (UAA, UGA, or UAG) appropriately positioned at the end of the polypeptide to be translated. The vector can also include appropriate sequences for amplifying expression.

As used herein, the phrase “operably linked” refers to a linkage in which a nucleotide sequence is connected to another nucleotide sequence (or sequences) in such a way as to be capable of altering the functioning of the sequence (or sequences). For example, a protein coding sequence which is operably linked to a promoter places expression of the protein coding sequence under the influence or control of this sequence. Two nucleotide sequences (such as a protein encoding sequence and a promoter region sequence linked to the 5′ end of the encoding sequence) are said to be operatively linked if induction of promoter function results in the transcription of the protein encoding sequence mRNA and if the nature of the linkage between the two nucleotide sequences does not (1) result in the introduction of a frame-shift mutation nor (2) prevent the expression of regulatory sequences to direct the expression of the mRNA or protein. Thus, a promoter region would be operably linked to a nucleotide sequence if the promoter were capable of effecting transcription of that nucleotide sequence. Two nucleotide sequences that are operably linked can also be linked through the use of a spacer molecule, i.e., they need not be immediately adjacent to each other.

As used herein, the phrase “cloning vector” refers to a plasmid or phage nucleic acid or other nucleic acid sequence which is able to replicate autonomously in a host cell, and which is characterized by one or a small number of endonuclease recognition sites at which such nucleic acid sequences may be cut in a determinable fashion without loss of an essential biological function of the vector, and into which nucleic acid may be spliced in order to bring about its replication and cloning. The cloning vector may further contain a selectable marker suitable for use in the identification of cells transformed with the cloning vector. Markers, for example, are puromycin, dihydrofolate reductase, erythromycin, ampicillin, and kanamycin resistance. The term “vehicle” is sometimes used for “vector.”

As used herein, the phrase “expression vector” refers to a vector similar to a cloning vector which is capable of expressing one or more structural genes cloned into the expression vector, after transformation of the expression vector into a host. In an expression vector, the cloned structural genes (any coding sequence of interest) are placed under the control of (i.e., operably linked to) certain sequences which allow such gene to be expressed in a specific host. Expression control sequences will vary, and may additionally contain transcriptional elements such as termination sequences and/or translational elements such as initiation and termination sites.

As indicated above, the expression vectors may include at least one selectable marker. Such markers include puromycin, dihydrofolate reductase or neomycin resistance for eukaryotic cell culture. Representative examples of appropriate hosts include, but are not limited to, insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

It will be appreciated by one of skill in the art that mammalian expression vectors may comprise one or more of the following: an origin of replication, a suitable promoter and enhancer, ribosome binding sites, a polyadenylation site, splice donor and acceptor sites, transcriptional termination sequences, and 5′ flanking nontranscribed sequences. Nucleic acid sequences derived from the SV40 splice, and polyadenylation sites can be used to provide the required nontranscribed genetic elements.

Expression vectors that may be used in the expression of the polypeptides of the invention in eukaryotes include pIND/hygro available from Invitrogen; pWLNEO, pSV2CAT, pOG44, pXT1 and pSG available from Stratagene; and pSVK3, pBPV, pMSG and pSVL available from Pharmacia; and NEOSPLA vectors and the like as set forth in U.S. Pat. Nos. 5,648,267, 5,733,779, 6,017,733, and 6,159,730, and other proprietary expression vectors.

In a further embodiment, the invention relates to host cells containing the above-described construct. The host cell can be a higher eukaryotic cell, such as a mammalian cell, or a lower eukaryotic cell. Introduction of the construct into the host cell can be effected by electroporation, calcium phosphate transfection, DEAE-dextran mediated transfection, cationic lipid-mediated transfection, transduction, infection or other methods. Such methods are described in many standard laboratory manuals, such as Davis et al., BASIC METHODS IN MOLECULAR BIOLOGY (1986).

Recombinant constructs may be introduced into host cells using well-known techniques such as infection, transduction, transfection, transvection, electroporation and transformation. The vector may be, for example, a phage, plasmid, viral or retroviral vector. Retroviral vectors may be replication competent or replication defective. In the latter case, viral propagation generally will occur only in complementing host cells.

Host cells are genetically engineered (transduced, transformed or transfected) with the vectors of this invention which can be, for example, a cloning vector or an expression vector. Host cells also refers to cells which were previously genetically engineered to express a polypeptide of interest, and are genetically engineered with a second expression vector or vector of the invention. The vector can be, for example, in the form of a plasmid, a viral particle, etc. The engineered host cells can be cultured in conventional nutrient media modified as appropriate for activating promoters, selecting transformants or amplifying XBP-1 and/or the polypeptide of interest. The culture conditions, such as temperature, pH, current, and the like, are those previously used with the host cell selected for expression, and will be apparent to the ordinarily skilled artisan.

The polynucleotides of the invention can be employed for producing XBP-1 and polypeptides of interest by recombinant techniques. Thus, for example, the polynucleotide sequences can be included in any one of a variety of expression vehicles, in particular vectors or plasmids for expressing a polypeptide. Such vectors include chromosomal, nonchromosomal and synthetic nucleic acid sequences, e.g., derivatives of SV40; vectors derived from combinations of plasmids, viral nucleic acid such as vaccinia, adenovirus, fowl pox virus, alphaviruses and pseudorabies. However, any other plasmid or vector can be used as long they are viable in the host.

As noted above, the vector containing polynucleotides encoding XBP-1, and optionally, additional polypeptides, and a selectable marker, as well as an appropriate promoter or control sequence, can be employed to transform an appropriate host to permit the host to express the protein.

As representative examples of appropriate hosts, there can be mentioned: insect cells such as Drosophila and Sf9; animal cells such as CHO, COS or Bowes melanoma; plant cells, etc. The selection of an appropriate host is deemed to be within the scope of those skilled in the art from the teachings herein.

The invention also includes recombinant constructs comprising one or more of the sequences as broadly described above. The constructs comprise a vector, such as a plasmid or viral vector, into which a sequence of the invention has been inserted, in a forward or reverse orientation. In an aspect of this embodiment, the construct further comprises regulatory sequences, including, for example, a promoter, operatively linked to the sequence. Large numbers of suitable vectors and promoters are known to those of skill in the art, and are commercially available. The following vectors are provided by way of example. Eukaryotic: pIND/hygro (Invitrogen), pWLNEO, pSV2CAT, pOG44, pXT1, pSG (Stratagene) pSVK3, pBPV, pMSG, pSVL (Pharmacia). However, any other plasmid or vector can be used as long as they are replicable and viable in the host.

Mature proteins can be expressed in mammalian or other cells under the control of appropriate promoters. Appropriate cloning and expression vectors for use with eukaryotic hosts are described by Sambrook, et al., Molecular Cloning: A Laboratory Manual, Second Edition, Cold Spring Harbor, N.Y., (1989), the disclosure of which is hereby incorporated by reference.

Various mammalian cell culture systems can be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. A “cell line” or “cell culture” denotes eukaryotic cells grown or maintained in vitro. It is understood that the descendants of a cell may not be completely identical (either morphologically, genotypically, or phenotypically) to the parent cell.

In addition to encompassing host cells containing the vector constructs discussed herein, the invention also encompasses primary, secondary, and immortalized host cells of vertebrate origin, particularly mammalian origin, that have been engineered to express endogenous genetic material (e.g. XBP-1 coding sequence), and/or to include genetic material (e.g. heterologous polynucleotide sequences) that is operably associated with XBP-1 polynucleotides of the invention, and which activates, alters, and/or amplifies endogenous XBP-1 polynucleotides. For example, techniques known in the art may be used to operably associate heterologous control regions (e.g., promoter and/or enhancer) and endogenous XBP-1 polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

For secretion of the translated polypeptide of interest into the lumen of the endoplasmic reticulum, or into the extracellular environment, appropriate secretion signals may be incorporated into the expressed polypeptide of interest. The signals may be endogenous to the polypeptide or they may be heterologous signals.

The polypeptide of interest may be expressed in a modified form, such as a fusion protein, and may include not only secretion signals but also additional heterologous functional regions. Thus, for instance, a region of additional amino acids, particularly charged amino acids, may be added to the N-terminus of the polypeptide to improve stability and persistence in the host cell, during purification or during subsequent handling and storage. Also, peptide moieties may be added to the polypeptide to facilitate purification.

The polypeptide of interest can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification.

Polypeptides of interest of the invention include naturally purified products, and products produced by recombinant techniques from a eukaryotic host, including, for example, yeast, higher plant, insect and mammalian cells. Depending upon the host employed in a recombinant production procedure, the polypeptides of the invention may be glycosylated or may be non-glycosylated. In addition, polypeptides of the invention may also include an initial modified methionine residue, in some cases as a result of host-mediated processes.

In one embodiment of the invention, host cells expressing the polypeptide of interest are mammalian cells. Examples of mammalian cells include monkey kidney CV1 line transformed by SV40 (COS-7, ATCC CRL 1651); human lens epithelium line PER.C6™ line; human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub and Chasin, Proc. Natl. Acad. Sci. USA, 77:4216 (1980)); monkey kidney cells (CV1 ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). CHO cells are a preferred cell line for practicing the invention.

Large-Scale Protein Expression

To generate large quantities of polypeptides of interest (e.g. antibodies) for use, it is desirable to employ an efficient recombinant expression system. Since myeloma cells represent a natural host specialized for antibody production and secretion, cell lines derived from these have been used for the expression of recombinant antibodies. Often, complex vector design, based around immunoglobulin gene regulatory elements, is required, and final expression levels have been reported which are highly variable (Winter et al., Nature 332:323-327 (1988); Weidle et al, Gene 60:205-216 (1987); Nakatani et al, Bio/Technology 7:805-10 (1989); and Gillies et al, Bio/Technology 7:799-804 (1989)).

An alternative mammalian expression system is that offered by the use of CHO cells. The use of these cells has enabled the production of large quantities of several therapeutic proteins for research and clinical use (Kaufman et al, Mol. Cell. Biol. 5:1750-1759 (1985); and Zettlmeissl et al, Bio/Technology 5:720-725 (1985)).

While in certain embodiments of the invention, specific cellular productivity of CHO cells expressing an antibody may be generally at the level of between 20-100 pg/cell/day, this range is not meant to be limiting. In some instances, the range may be greatly reduced such as under large-scale manufacturing conditions. Other embodiments of the invention may result in specific cellular productivity of 5 pg/cell/day or even less provided the proteins are produced by the methods of the invention. In various embodiments of the invention, the method results in increases in the specific cellular productivity of a polypeptide relative to control cells previously transfected with the polypeptide of interest without XBP-1 of about 5% to about 10%, about 11% to about 20%, about 21% to about 30%, about 31% to about 40%, about 41% to about 50%, about 51% to about 60%, about 61% to about 70%, about 71% to about 80%, about 81% to about 90%, or about 91% to about 100%. Thus, the culturing process enables increased quantities of functional antibody to be obtained for example, for use in the immunotherapy of pathological disorders.

The polypeptides of interest of the invention may be produced by growing cells which express the desired polypeptide under a variety of cell culture conditions. For instance, cell culture procedures for the large- or small-scale production of polypeptides are potentially useful within the context of the invention. Procedures including, but not limited to, a fluidized bed bioreactor, hollow fiber bioreactor, roller bottle culture, or stirred tank bioreactor system may be used, in the later two systems, with or without microcarriers, and operated alternatively in a batch, fed-batch, or continuous mode.

In one embodiment the cell culture of the invention is performed in a stirred tank bioreactor system and a fed-batch culture procedure is employed. In fed-batch culture, the mammalian host cells and culture medium are supplied to a culturing vessel initially and additional culture nutrients are fed, continuously or in discrete increments, to the culture during culturing, with or without periodic cell and/or product harvest before termination of culture. The fed-batch culture can include, for example, a semi-continuous fed-batch culture, wherein periodically whole culture (including cells and medium) is removed and replaced by fresh medium. Fed-batch culture is distinguished from simple-batch culture in which all components for cell culturing (including the cells and all culture nutrients) are supplied to the culturing vessel at the start of the culturing process. Fed-batch culture can be further distinguished from perfusion culturing insofar as the supernate is not removed from the culturing vessel during the process (in perfusion culturing, the cells are restrained in the culture by, e.g., filtration, encapsulation, anchoring to microcarriers, etc., and the culture medium is continuously or intermittently introduced and removed from the culturing vessel).

Further, the cells of the culture may be propagated according to any scheme or routine that may be suitable for the particular host cell and the particular production plan contemplated. Therefore, the invention contemplates a single-step or multiple-step culture procedure. In a single-step culture the host cells are inoculated into a culture environment and the processes of the instant invention are employed during a single production phase of the cell culture. Alternatively, a multi-stage culture is envisioned. In the multi-stage culture cells may be cultivated in a number of steps or phases. For instance, cells may be grown in a first step or growth phase culture wherein cells, possibly removed from storage, are inoculated into a medium suitable for promoting growth and high viability. The cells may be maintained in the growth phase for a suitable period of time by the addition of fresh medium to the host cell culture.

According to one aspect of the invention, fed-batch or continuous cell culture conditions are devised to enhance growth of the mammalian cells in the growth phase of the cell culture. In another aspect, a bulk cell culture method is devised for cell growth. During fed-batch, or continuous cell culture conditions, the growth phase cells are grown under conditions and for a period of time that is maximized for growth. Culture conditions, such as temperature, pH, dissolved oxygen (DO₂), and the like, are those used with the particular host and will be apparent to the ordinarily-skilled artisan. The pH is adjusted to an appropriate level generally between about 6.5 and 7.5 using either an acid (e.g., CO₂) or a base (e.g., Na₂CO₃ or NaOH). A suitable temperature range for culturing mammalian cells such as CHO cells is between about 30 to 40° C. and generally about 37° C. and a suitable DO₂ is between 5-90% of air saturation. However, higher or lower ranges for any of these conditions may be necessary depending on the cell type.

At a particular stage the cells may be used to inoculate a production phase or step of the cell culture. Alternatively, as described above the production phase or step may be continuous with the inoculation or growth phase or step.

According to the invention, the cell-culture environment during the production phase of the cell culture is controlled. In one aspect, the production phase of the cell culture process is preceded by a transition phase of the cell culture in which parameters for the production phase of the cell culture are engaged.

Antibody production in mammalian, e.g., CHO, cells may employ a semi-continuous process whereby cells are cultured in a “seed-train” for various periods of time and are periodically transferred to inoculum fermentors to initiate the cell-amplification process en route to larger scale production of the polypeptide of interest. Thus, cells used for antibody production are in culture for various periods of time up to a maximum predefined cell age. The parameters of the cell culture process, such as seed density, pH, DO₂ and temperature during culture, duration of the production culture, operating conditions of harvest, etc. are a function of the particular cell line and culture medium used, and can be determined empirically, without undue experimentation.

### EXAMPLES

### Example 1

Enhanced Secretion Cell Lines

In an effort to alleviate product supply constraints and reduce manufacturing costs, several strategies to improve the antibody yields of manufacturing cell lines were evaluated. One strategy is to incorporate secretion enhancing genes into the genetic constitution of production cell lines.

It has previously been demonstrated that overexpression of individual chaperones and foldases can facilitate proper folding and assembly of secreted polypeptides. However, in most of these cases, the effect of the chaperone or foldase overexpressed is polypeptide specific. One possible way to improve secretion of a recombinant host, irrespective of the protein to be produced, may be to upregulate expression of a large number of genes involved in the secretion process through activation of the UPR.

In short, the UPR is a response to the accumulation of unfolded polypeptides in the ER of eukaryotic cells. Once stimulated, the UPR can manage the accumulation of unfolded/misfolded polypeptides in one or more of the following ways: (1) upregulate multiple components of the secretory apparatus; (2) attenuate polypeptide translation; and (3) retro-translocate polypeptides for subsequent degradation.

In an attempt to enhance antibody secretion, several genes have been overexpressed in a single-cell isolate, 500 nM G418-amplified cell line expressing an anti-CD154 antibody, to upregulate components of the secretory apparatus and to overcome translation attenuation. These genes are a spliced form of XBP-1, nuclear ATF6, and the eIF2α S51A mutant.

To address upregulation of secretory components, XBP-1 (SEQ ID NO:2) and XBP-1 in concert with ATF6 (SEQ ID NO:4) was expressed.

To address translation attenuation, eIF2α S51A mutant (SEQ ID NO:6) was expressed in concert with XBP-1 and nuclear ATF6. During the UPR, polypeptide synthesis may be reduced due to events that result in phosphorylation of the translation initiation factor eIF2α. The mutation in eIF2α prevents phosphorylation of a key residue in the protein that is responsible for its role in translation attenuation.

Cell Line Construction

500E9 is a production cell line that produces a humanized recombinant anti-CD154 monoclonal antibody. 500E9 is used for purposes of illustration and should not be viewed as limiting. Any cell line, such as those mentioned previously, expressing a polypeptide of interest may be used. The 500E9 cell line was obtained following three successive rounds of methotrexate amplification (5 nM, 50 nM, and 500 nM) after an initial G418 selection. 500E9 cells were generated by initially transfecting CHO DG44 cells with a plasmid DNA expression vector containing the anti-CD154 expression sequence and the G418 selectable marker to create the G418 clone. 500E9 cells were then electroporated with S8 plasmid DNA that contains the eIF2α mutant, nuclear ATF6, and XBP-1 genes as well as the puromycin resistance gene, resulting in generation of the S8-7E4 cell line. Following electroporation, S8-7E4 was selected for puromycin resistance from a 96 well plate and expanded. SX11 (eIF2α-mutant and XBP-1) and NSX7 (XBP-1) plasmid DNA was also used to electroporate 500E9 cells. Both the SX11-9E8 And NSX7-1C9 cell lines were selected for puromycin resistance from 96 well plates. The 500E9 cells used to generate the modified stable cell lines were obtained no longer than 8 weeks prior to electroporation.

The plasmid incorporating the secretion enhancing genes was modified from the pIND/hygro vector (Invitrogen). The new vector contains a puromycin resistance gene, a multiple expression cassette, transcription terminators, mouse betaglobin promoters, and a sea urchin transcription insulator (SNS).

Results

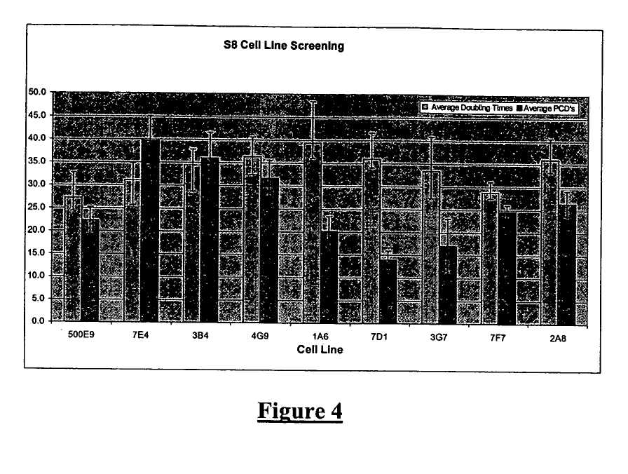

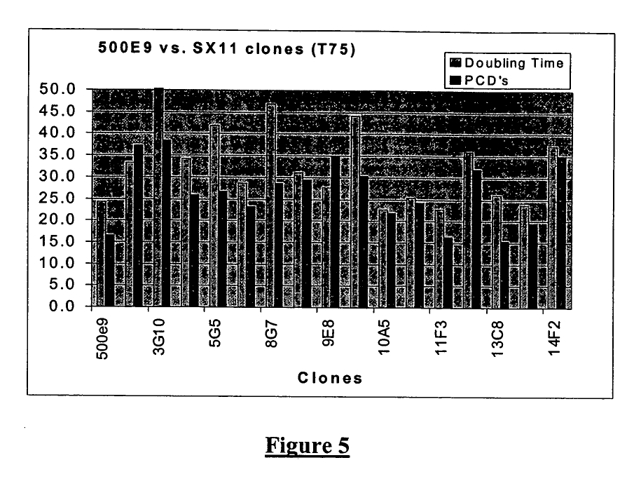

The cell lines were initially screened for potential candidates by either spinner flask or T-flask. In spinner flasks, cell lines were evaluated for doubling time (DT) and specific productivity (SPr) in three and four day cultures (FIG. 4). SPr and growth rate was averaged from a minimum of three such spinners. In T-flasks, SPr and growth rate was determined from a single five-day culture (FIG. 5). The T-flasks were employed to examine a larger number of cell lines in a shorter period of time than using the spinner flasks. A total of 16 S8, 22 SX11, and 25 NSX7 cell lines were screened. Cell lines that had a higher specific productivity and had an equivalent doubling time to 500E9 were kept for further study. The others were eliminated.

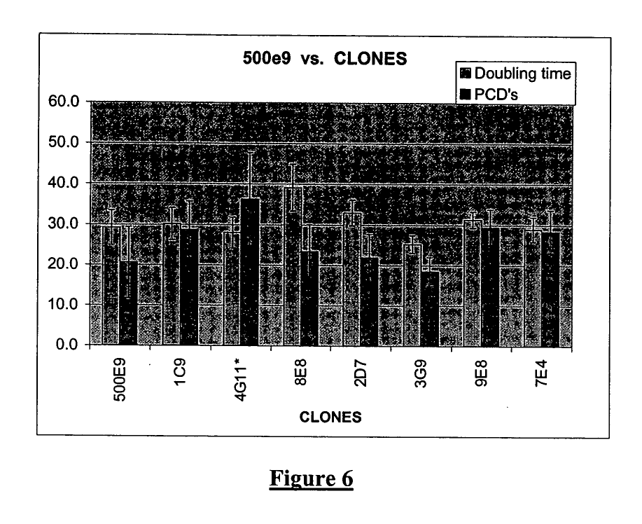

After the initial screening, 5 S8, 6 SX11, and 6 NSX7 cell lines were evaluated in a minimum of 1 duplicate and 1 triplicate spinner flask experiment. Spinner flask cultures were seeded at 2e⁵ or 3e⁵ cells/ml in fresh medium on day zero. Samples were withdrawn daily, counted, and analyzed for antibody levels (as determined by ELISA). The DT and SPr were determined from the average of those values from days 2 to 4 of the culture period. From examining and compiling all the data, cell lines S8-7E4, SX11-9E8, and NSX7-1C9 were the best performers from each of the cell line constructs. When compared to 500E9, the results from the final spinner flask evaluation show a 35%, 43%, and 39% increase in SPr for cell lines S8-7E4, SX11-9E8, and NSX7-1C9, respectively (FIG. 6). Thus, modification of the UPR by increasing expression levels of XBP-1, nuclear ATF6, and eIF2α S51A, either individually, or in combination, resulted in an increase in the specific cellular productivity of the anti-CD154 humanized antibody.

Single cell isolates of any new production cell line can be obtained by selecting cell lines that contain a secretion enhancing construct at a single integration site in the host's genome. Selection of a single cell isolate using a secretion enhancing construct can improve production without requiring any more time than the current single cell isolation process.

The foregoing specification, including the specific embodiments and examples, is intended to be illustrative of the invention and is not to be taken as limiting. Numerous other variations and modifications can be effected without departing from the true spirit and scope of the invention. All publications, patents and patent applications cited herein are incorporated by reference in their entirety into the disclosure.

1. A method for increasing specific cellular productivity of a membrane or secreted polypeptide of interest in a cell, said method comprising expressing said polypeptide of interest, wherein said cell also expresses an exogenous, spliced form of XBP-1.

2. The method of claim 1, wherein a polynucleotide encoding said polypeptide of interest is exogenous.

3. The method of claim 1, wherein said method results in increased specific cellular productivity of said polypeptide of interest relative to control cells previously transfected with the polypeptide of interest without XBP-1, said increase selected from the group consisting of: about 5% to about 10%, about 11% to about 20%, about 21% to about 30%, about 31% to about 40%, about 41% to about 50%, about 51% to about 60%, about 61% to about 70%, about 71% to about 80%, about 81% to about 90%, and about 91% to about 100%.

4. The method of claim 1, wherein said cell further expresses a selectable marker.

5. The method of claim 4, wherein said method further comprises detecting cells expressing said polypeptide of interest by detecting integration of the selectable marker.

6. The method of claim 1, wherein said method results in the production of the polypeptide of interest at a level of at least about 5 pg/cell/day.

7. The method of claim 1, wherein said XBP-1 is encoded by the polynucleotide of SEQ ID NO:1.

8. The method of claim 1, wherein said XBP-1 is encoded by a polynucleotide encoding the polypeptide of SEQ ID NO:2.

9. The method of claim 1, wherein said cell further expresses an additional polynucleotide.

10. The method of claim 9, wherein said additional polynucleotide encodes a polypeptide which is a second transcription factor or a translation enhancer.

11. The method of claim 10, wherein said second transcription factor is ATF6 or its activation domain.

12. The method of claim 11, wherein said ATF6 is encoded by a polynucleotide encoding the polypeptide of SEQ ID NO:4.

13. The method of claim 10, wherein said translation enhancer is eIF2α S51A.

14. The method of claim 13, wherein said eIF2α S51A is encoded by a polynucleotide encoding the polypeptide of SEQ ID NO:6.

15. The method of claim 1, wherein said polypeptide of interest is an antibody or antibody fragment.

16. The method of claim 15, wherein said antibody is selected from the group consisting of: monoclonal, polyclonal, mammalian, murine, chimeric, humanized, primatized, primate, and human.

17. The method of claim 16, wherein said antibody is a monoclonal antibody.

18. The method of claim 16, wherein said antibody is chimeric.

19. The method of claim 15, wherein said antibody is the anti-CD154 antibody Hu24-31.1.

20. The method of claim 15, wherein said antibody fragment is selected from the group consisting of: immunoglobulin light chain, immunoglobulin heavy chain, immunoglobulin light and heavy chains, Fab, F(ab′)₂, Fc, Fc-Fc fusion proteins, Fv, single-chain Fv, tetravalent single chain Fv, disulfide-linked Fv, domain deleted, minibody, diabody, and a fusion polypeptide of one of the above fragments with another polypeptide.

21. The method of claim 20, wherein said domain deleted antibody fragment is a C_{H}2 domain deleted antibody fragment.

22. The method of claim 1, wherein said cell is a mammalian cell.

23. The method of claim 1, wherein said cell is an eukaryotic cell.

24. The method of claim 1, wherein said cell is an insect cell.

25. The method of claim 22, wherein said mammalian cell is a chinese hamster ovary cell.

26. The method of claim 22, wherein said mammalian cell is selected from the group consisting of: monkey kidney CV1, monkey kidney COS, human lens epithelium PER.C6™, human embryonic kidney, baby hamster kidney, african green monkey kidney, human cervical carcinoma, canine kidney, buffalo rat liver, human lung, human liver, mouse mammary tumor cells, and myeloma cell lines.

27. The method of claim 26, wherein said myeloma cell lines are selected from the group consisting of NS0, Sp2/0, and Ag8653.

28. A method of increasing specific cellular productivity of a membrane or secreted polypeptide of interest in a cell, said method comprising introducing into a cell a vector comprising two polynucleotides, wherein said first polynucleotide encodes the spliced form of the transcription factor XBP-1 and said second polynucleotide encodes a polypeptide of interest, and expressing said polypeptide of interest.

29. An expression vector comprising a first polynucleotide and a second polynucleotide, wherein said first polynucleotide comprises a polynucleotide which encodes a spliced form of the transcription factor XBP-1 and said second polynucleotide comprises a polynucleotide which encodes a selectable marker.

30. The expression vector of claim 29, wherein said spliced form of XBP-1 is encoded by the polynucleotide of SEQ ID NO:1.

31. The expression vector of claim 29, wherein said spliced form of XBP-1 is 95% identical to SEQ ID NO:1, and said vector produces an increased specific cellular productivity of a membrane or secreted polypeptide of interest when said vector is introduced into a cell expressing a polypeptide of interest.

32. The expression vector of claim 29, wherein said first polynucleotide comprises nucleotides 195 to 356 of SEQ ID NO:1, wherein said nucleotides 195 to 356 produce an increased specific cellular productivity of a membrane or secreted polypeptide of interest when introduced into a cell.

33. The expression vector of claim 29, wherein said first polynucleotide encodes the polypeptide of SEQ ID NO:2.

34. The expression vector of claim 29, wherein said expression vector further comprises an additional polynucleotide.

35. The expression vector of claim 34, wherein said additional polynucleotide encodes a polypeptide which is a second transcription factor or a translation enhancer.

36. The expression vector of claim 35, wherein said second transcription factor is ATF6 or its activation domain.

37. The expression vector of claim 36, wherein said ATF6 is encoded by a polynucleotide encoding the polypeptide of SEQ ID NO:4.

38. The expression vector of claim 35, wherein said translation enhancer is eIF2α S51A.

39. The expression vector of claim 38, wherein said eIF2α S51A is encoded by a polynucleotide encoding the polypeptide of SEQ ID NO:6.

40. A cell comprising the expression vector of claim 29.

41. The cell of claim 40, wherein said cell is an animal cell.

42. The cell of claim 40, wherein said cell is an eukaryotic cell.

43. The cell of claim 40, wherein said cell is an insect cell.

44. The cell of claim 40, wherein said cell is a mammalian cell.

45. The cell of claim 44, wherein said mammalian cell is a chinese hamster ovary cell.

46. The cell of claim 44, wherein said mammalian cell is selected from the group consisting of: monkey kidney CV1, monkey kidney COS, human lens epithelium PER.C6™, human embryonic kidney, baby hamster kidney, african green monkey kidney, human cervical carcinoma, canine kidney, buffalo rat liver, human lung, human liver, mouse mammary tumor, and myeloma cell lines.

47. The cell of claim 46, wherein said myeloma cell lines is selected from the group consisting of NS0, Sp2/0, and Ag8653.

48. The expression vector of claim 29, wherein said first polynucleotide upregulates the unfolded protein response in a cell.

49. An isolated polynucleotide comprising a first component encoding the spliced form of XBP-1 and a second component encoding a selectable marker, wherein said isolated polynucleotide, when introduced into a cell expressing a polypeptide of interest, produces an increased specific cellular productivity of a polypeptide of interest of at least 5 pg/cell/day in said cell.

50. The isolated polynucleotide of claim 49, wherein said isolated polynucleotide further comprises a polynucleotide encoding a polypeptide of interest.

51. The isolated polynucleotide of claim 49, wherein said spliced form of XBP-1 is encoded by a polynucleotide encoding the polypeptide of SEQ ID NO:2.

52. The isolated polynucleotide of claim 49, wherein said first component further comprises an additional polynucleotide.

53. The isolated polynucleotide of claim 52, wherein said additional polynucleotide encodes a polypeptide which is a second transcription factor or a translation enhancer.

54. The isolated polynucleotide of claim 53, wherein said second transcription factor is ATF6 or its activation domain.

55. The isolated polynucleotide of claim 54, wherein said ATF6 is encoded by the polynucleotide of SEQ ID NO:3.

56. The isolated polynucleotide of claim 53, wherein said translation enhancer is eIF2α S51A.

57. The isolated polynucleotide of claim 56, wherein said eIF2α S51A is encoded by the polynucleotide of SEQ ID NO:5.

58. An antibody or antibody fragment made by the method of claim 1.

59. A method of increasing specific cellular productivity of a membrane or secreted polypeptide of interest in a cell, said method comprising introducing into a cell expressing a polypeptide of interest, an isolated polynucleotide comprising a first component encoding the spliced form of XBP-1 and a second component encoding a selectable marker, wherein said isolated polynucleotide produces an increased specific cellular productivity of said polypeptide of interest of at least 5 pg/cell/day in said cell.

60. The method of claim 1, wherein said cell further expresses one or more additional polynucleotides.

61. The method of claim 60, wherein said one or more additional polynucleotides encodes a polypeptides which is a second transcription factor and a translation enhancer.
